(19) United States
(12) Patent Application Publication
(10) Pub. No.: US 2017/0215769 A1
(43) Pub. Date: Aug. 3, 2017

(54) APPARATUS AND A METHOD FOR DETECTING THE POSTURE OF THE ANATOMY OF A PERSON

(71) Applicant: SHENZHEN XIAO LUO JI TECHNOLOGY LIMITED, Shenzhen (CN)

(72) Inventors: Shan Lu, Choa Chu Kang (SG); Meichen Lu, Cambridge (GB)

(73) Assignee: SHENZHEN XIAO LUO JI TECHNOLOGY LIMITED, Shenzhen (CN)

(21) Appl. No.: 15/514,990

(22) PCT Filed: Oct. 7, 2015
(86) PCT No.: PCT/IB2015/001817

§ 371 Date: Mar. 28, 2017

(30) Foreign Application Priority Data
Oct. 7, 2014 (GB) 1417721.6

(51) Int. Cl. A61B 5/11; A61B 5/00

(52) U.S. Cl. CPC A61B 5/1116; A61B 5/1121; A61B 5/6822; A61B 5/746; A61B 5/0022

(57) ABSTRACT

An apparatus can be used in the detection and correction of the posture of a person's anatomy and particularly, although not necessarily exclusively, the posture of a person's neck; The apparatus includes a device to be worn, such as at the rear of the neck of a person, and the device includes a frame having first and second ends and at least one sensor to detect the tilt angle of a predefined plane and processing means to determine whether the detected tilt angle is within a predetermined range of acceptable positions; The apparatus, when worn, has a plurality of contact points with the wearer.

The present invention relates to apparatus for use in the detection and correction of the posture of a person's anatomy and particularly, although not necessarily exclusively, the posture of a person's neck.

Muscle strain is a common problem experienced by most persons, permanently or on an occasional basis. In many cases, the muscle strains can be in specific areas of the body, such as those which cause neck pain, and the triggers for this type of pain can be traced to one or several common activities. One such activity can be the person adopting an incorrect sitting position for a prolonged period of time, such as hunching over a steering wheel while driving, leaning forward to view a computer monitor, or cradling a phone with a crooked neck. Studies have shown that the risk of neck pain for workers and school children who sit for more than 95% of their working time increases significantly.

It is known that there are an optimal range of head positions and neck inclinations within which the force from one or more muscles can balance the weight of the head with minimal strain and hence reduce the risk of neck pain. It is therefore found that individuals whose head positions lie within this optimal range tend to enjoy healthy sitting postures and avoid suffering from neck pain. In addition these individuals appear to be generally more confident when the correct sitting positions are adopted. Studies have also shown that this increased confidence can also materially improve the person's mental status and behaviour. However, the adoption of head positions outside of this optimal range requires a significantly higher force to be used to support the head and neck in these less favourable positions, if this high level of force requirement and hence stress is maintained over a prolonged period of time, symptoms of neck strain and discomfort can be caused in the short term and can lead to progressive deterioration and possible chronic spine disease in the longer term.

Devices that aim at measuring range of motion and back posture are shown in U.S. Pat. No. 5,337,759 A and U.S. Pat. No. 4,928,709 A, both of which comprise a frame supported on top of the wearer's head. Other devices that aim at posture correction or detection, such as U.S. Pat. No. 5,425,378A and U.S. Pat. No. 5,337,759A, determine the posture of the users solely by sensing and recording the inclination of the head. However this is not always reliable because one major category of incorrect posture, that known as “forward head”, which is when the neck slants forward placing the head in front of the shoulders so that there is no detectable inclination, cannot be detected by this factor alone. This problem, and the accuracy of any detection is made worse by the provision of only one contact point of the known devices and the body of the wearer.

The aim of the present invention is therefore to provide apparatus and a method for detecting the location of a person's head and identifying whether the detected position is within or outside a predetermined range of acceptable positions. A further aim is to minimise the occurrences of the head position being incorrect.

In a first aspect of the invention there is provided apparatus including a frame having location means, at least one sensor to detect the tilt angle of a plane which is representative of the tilt angle of the head of the person with respect to their neck when wearing the apparatus and processing means which receives data from the at least one sensor which is representative of the detected tilt angle to determine whether the detected tilt angle is within a predetermined range of acceptable positions and wherein the apparatus has a plurality of contact points with the person when worn.

In one embodiment the respective ends of the frame are located so as to provide a stable relatively fixed position of the frame and a contact point at the location of each of ears of the person.

Typically the one or more sensors can be selectively positioned at any desired position with respect to the frame and still provide an indication of the said plane with respect to an optimal or desired plane. The provision of the measurement of the tilt angle of the plane means that there isn't a need to provide several “sampling points”, as, as long as the sensor is in a reproducible relative position with respect to the frame.

Typically the frame has at least 3 reproducible contact points with the user's anatomy this, in turn, forms a reproducible virtual plane and it is the tilt of the plane that the sensor is measured.

In one embodiment, the apparatus includes alert means to notify the person and/or a third party when the detected tilt angle is outside the said predetermined range.

In one embodiment the frame has two opposing contact points with the person at a stable location. In one embodiment these stable locations are at the person's ears. Typically a third contact point is provided intermediate the two opposing contact points. Typically the third contact point is located at the rear of the head or neck of the person.

In one embodiment the said at least one sensor is mounted substantially centrally of the frame.

In one embodiment the apparatus includes a power supply to allow operation of the sensors.

In one embodiment the processing means include an integrated circuit with a micro controller unit which processes information from the said sensors to generate the detected tilt angle, compare the same with reference to predetermined data and means to generate corresponding responses, typically with reference to the predetermined data.

In one embodiment the processing means includes a wireless radio frequency transmitter which outputs responses from the micro controller unit.

In one embodiment the processing means includes, or is connected to, an alarm unit which can be used to generate audible, visual and/or vibrational signals.

In one embodiment the apparatus includes means to track and analyse collected data from the one or more sensors.

In one embodiment the frame supports and positions the one or more sensors to the rear of the head and/or neck of the user, with opposing ends of the frame being secured over the ears of the person wearing the device.

In one embodiment the sensors are provided in order to allow the head inclinations and neck-flexions on orthogonal axes to be determined and monitored.

In one embodiment the processing means can provide different types of feedback, which, in one embodiment, can be selected to suit a person's and/or a third party's requirements, and these may include any, or any combination of; an instant or delayed reminder when the user has adopted a posture with inclinations and/or neck-flexions which lie outside the said predetermined range in order to cause the person to attempt to rectify the incorrect position, and/or provide positive confirmation when the person is detected as having adopted a position within the predetermined range in order to assist them in maintaining the new, acceptable position.

Typically the apparatus can be used to provide medical support, such as to prevent spinal disease by ensuring that the person adopts the correct posture and/or assists in the confidence of the person by allowing the person to adopt a correct sitting posture.

The invention therefore provides apparatus which can be used as a health and/or beauty product with an analysis system to monitor neck postures. As the device contains a combination of sensors which provide feedback based on the recorded information of neck and head positions and movements of the wearer the data representative of this can be fed to the processing means which process the same via in-built algorithms in the MCU and the data is analysed with reference to personal data of the wearer to provide customised feedback. Personal feedback can be presented in various forms such as instant notification and/or transmission of data for further analysis, in order to monitor, rectify and improve the wearer's postures relating to head, neck and shoulder.

The present invention also has the advantage of achieving more reliable and reproducible results in comparison to the results which are provided from prior art apparatus which typically have insufficiently stable reference points as, if the device is not properly secured, measurement errors are likely to result. The provision of the frame and the plane which this allows to be generated, ensures measurements of the angle of orthogonal axes from the apparatus of the current invention are accurate and reproducible.

The apparatus of the present invention can be used to prevent and ameliorate a comprehensive range of discomfort and chronic neck disease resulting from incorrect adopted postures. The present invention is able to detect head deviations from the tilt angles in a plurality of axes, which serves as an improvement from previous detection measures. The invention sends out instant and/or delayed notifications to the user when rectifications of postures are recommended. Therefore, it monitors the person's behaviour to both prevent neck disease and improve aesthetic appearance of the user. The stabilised head-neck plane minimizes the risk of inaccuracies resulting from incorrect wearing of the device and ensures that measurements are reproducible.

Specific embodiments of the invention are now described with reference to the accompanying Figures; wherein

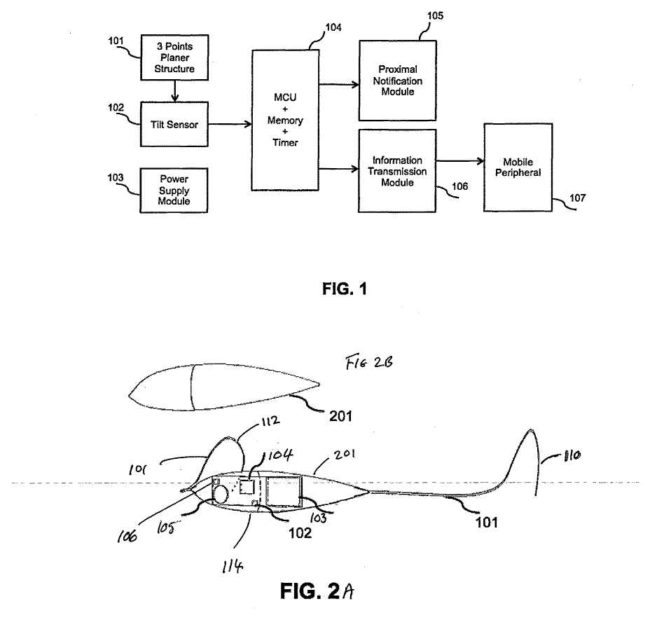

FIG. 1 illustrates a flow diagram of the invention in accordance with one embodiment of the invention;

FIG. 2a illustrates the components of the apparatus of the invention in accordance with one embodiment;

FIG. 2b illustrates the third contact point housing of the apparatus of FIG. 2a in one embodiment;

FIG. 2c shows the apparatus being worn for use; and

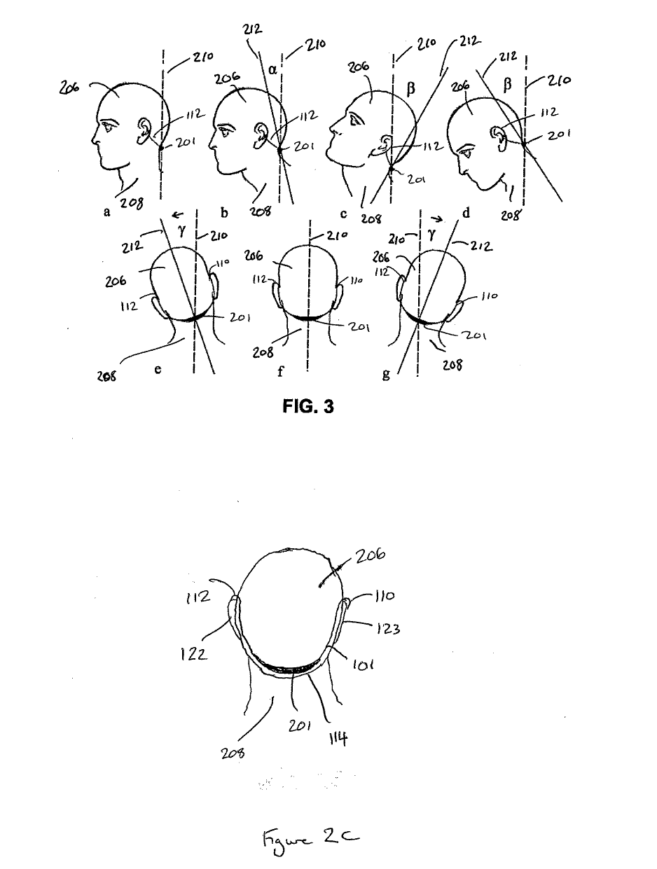

FIG. 3 shows profiles of a person wearing the device of FIG. 2 and various effective postures which can be detected by the device.

The apparatus in accordance with one embodiment of the invention is shown in FIGS. 2a-c and is worn by the person whose posture is to be detected. The apparatus includes a frame 101 with two opposing ends 110, 112 which act as contact points with the person. In one embodiment the ends are bent as shown into loops to allow the same to be located on the respective ears of the person so that the frame 101 is worn and supported behind the head and/or neck of the person when in use. FIG. 2c and FIG. 3 illustrate the frame 101 when worn and the frame is designed with opposing ends which act as contact points 110, 112. The ends rest on, and typically wrap partially around, the ears 122, 123 of the wearer's head 206 to hold the frame in a relatively fixed position with respect to the person's head as shown in FIG. 2c and FIG. 3. A housing 114 forms another point of contact 201 with the person, and in which at least one sensor can be located. The housing 114 is positioned and rests against the rear of the neck 208 or head 206 of the person, and typically at the interface between the head and neck as shown. Preferably the material of the frame is sufficiently resilient so as to maintain its shape during motion and also to ensure that the frame is the same shape upon repeated use and therefore the sensors, which are typically enclosed and protected by a plastic cover, are provided at the same position upon each use. Thus the provision of the multiple contact points and the provision of the apparatus in the same position upon each wear means that the data results which are obtained upon each use tend to be for the same or a similar position of the sensor. This, in turn, means that comparison of the data with data obtained from previous uses of the apparatus, are in relation to the same wearing position on the person and therefore gives the comparison greater validity. The same also applies to the comparison with predetermined data as the user can have greater certainty that the sensor and the apparatus is being worn in the correct position and the position to which the predetermined data relates. This is in contrast to conventional posture apparatus which are not worn at all or may be worn on a necklace or applied to the wearer, and which means that the sensor position cannot be accurately predicted during a single use or the sensor may be positioned at a different location on the person at different times of use.

The third contact point 114 is provided as part of the housing 201 which, as shown, is located intermediate the first and second contact points 110, 112 and FIG. 2a shows the housing 201 with a cover removed and FIG. 2b shows the housing in the form for use in one embodiment.

The three contact points 110,112,114 create a virtual plane and the at least one tilt detecting sensor is provided to measure the relative angular or tilt position of the plane. In this case the tilt sensor (102) is located at the third contact point housing 201 at the rear of the neck of the wearer and this sensor measures the angle of tilt of the plane formed by the three contact points 110,112, 114.

The apparatus also includes a central MCU with an inbuilt clock (104) which processes the measured data and recognises patterns and numbers that correspond to strained muscle and other incorrect postures.

A proximal information notification module is also provided which sends reminders when the user's neck deviation is beyond the optimal range to help the user attain the correct postures. The system can also be personally customised to send the user positive confirmation when they have assumed the correct posture for a period of time so as to help them maintain the recommended sitting position.

It is also possible for the data to be transmitted to a peripheral device, e.g. a smart phone for further recording and analysis to track the health development of the user and in this case an information transmission module (106), typically wireless based, can be used to relay the information to a peripheral device, e.g. a mobile phone of the user directly.

The data flow and processing in accordance with the invention is illustrated in FIG. 1 and illustrates at block 101 the three contact points, 110, 112, 114 which are located, two at the ears of the wearer and one at the back of the neck. The one or more tilt sensors 102 are used to detect the tilt angle of the plane formed between these three contact points and with respect to two different axes. A power supply 103 is provided which, in this case, is a small battery to supply working voltage to the electronic timer/controller integrated circuits. The integrated circuit 104 contains a micro controller unit (MCU) which in turn includes a built-in memory and clock. The device can alert the person wearing the device if an incorrect position has been held for a certain time period via the proximal notification module 105 which can emit audible (speaker) and/or motional (vibrator) alert.

Information can also be transmitted via a wireless radio frequency (RF) transmitter 106 (e.g. Bluetooth) to a mobile peripheral item. The alert can therefore be achieved via the interface (including visual, audible and/or motional signals) of the mobile peripheral. Additionally, the information can be logged in the mobile peripheral and can be accessed over a period of time as a tool for tracking the user history.

A battery supplies power to the electronic circuits and an external, charging power supply from the grid with an AC to DC converter can also be provided.

The tilt sensors 102 detect the signal of the angle of rotation of the plane from the vertical axis and the data can be reported with respect to two planar directions, which typically will be in the sagittal plane and the coronal plane.

The generation of alerts based on the analysis of the data can be achieved using audible alerts such as those generated by one or more speakers which can generate a beeping, atonal, music or voice sound to alert the user and/or via motional alerts in which a motor 204 can be used to generate a vibration to alert the user. In addition, or alternatively, a wireless alert signal can be transmitted to a peripheral device, e.g. mobile phone.

The processing means include the MCU 104 which implements the algorithms that process input and generate output and include sensor input circuit, timer circuitry, an alert function and driver circuit for vibrator, speaker.

FIG. 3 illustrates the various head and neck positions which can be detected using the apparatus in accordance with the invention and FIGS. 3a-d illustrate side views of the head 206 and neck 208 of the wearer of the apparatus

In FIGS. 3a and f the plane 210 is the normal plane and is the plane against which the detected tilt angles are subsequently determined.

In FIG. 3b a forward head tilt angle α of the detected plane 212 with respect to the normal plane 210 is identified.

In FIG. 3c the neck flexion up and in FIG. 3d the neck flexion down is detected by the sensor 102 such that the inclination angle of the neck 208 is detected in which β: the tilt angle of the head in the sagittal plan of the detected plane 212 with respect to the plane 210 is determined.

In FIG. 3e neck-flexion to the left is determined and in FIG. 3g neck-flexion to the right is determined and the tilt angle γ of the head in the coronal plane is determined.

The determination of these three angles with respect to the normal plane describe the entire neck posture at an instant of time and are used for further correction or assessment by determination of whether they lie within a predetermined range of acceptable positions.

With the angle of variation of the measured plane 212 being compared to the preferred or optimal plane 210 in order to be able to gauge whether or not the measured plane, and hence the posture of the person, is within acceptable limits.

The apparatus of the current invention therefore ensures that accurate measurements of the position of the apparatus wearer's head and/or neck with respect to the remainder of their body can be accurately detected. Furthermore as the apparatus has a frame with a plurality of contact points with the person, such as at the ears and the back of the neck, then the wearer can be more certain that the apparatus will be worn in the same position on the head/neck upon each time the frame is worn. This is due to the locating means at the respective ears which ensure that the relative position of the apparatus, and in particular the sensor, is substantially the same upon each time of wearing of the apparatus by the person.

1. Apparatus including a frame having location means, at least one sensor to detect the tilt angle of a plane which is representative of the tilt angle of the head of a person with respect to their neck when wearing the apparatus and processing means which receives data from the at least one sensor which is representative of the detected tilt angle to determine whether the detected tilt angle is within a pre-determined range of acceptable positions and the apparatus has a plurality of contact points with the person, when worn, and wherein at least one sensor is capable of detecting the angle of the head in a forward direction with respect to the person's neck.

2. Apparatus according to claim 1 wherein the said plane is formed between three or more points

3. Apparatus according to claim 1 wherein the respective ends of the frame are located so as to provide a stabilised position at the person's ears.

4. Apparatus according to claim 3 wherein a contact point is provided between the frame and the person at each ear.

5. Apparatus according to claim 1 wherein the sensor is selectively positionable at any desired position on the device to provide an indication of the said plane with respect to an optimal or desired plane.

6. Apparatus according to claim 1 wherein the frame has at least 3 reproducible contact points with the person.

7. Apparatus according to claim 5 wherein the sensor is located in a fixed position to the frame and the tilt of the plane is measured by the sensor.

8. Apparatus according to claim 1 wherein the apparatus includes alert means to notify the person and/or a third party when the detected tilt angle is outside of the said predetermined range.

9. Apparatus according to claim 1 wherein the device includes a frame with two opposing contact points with the person at or adjacent to the ears of the person wearing the apparatus, and a third intermediate contact point at which contact is made between the frame and the rear of the head or neck of the person when the apparatus is worn.

10. Apparatus according to claim 1 wherein the sensor is mounted substantially centrally of the frame.

11. Apparatus according to claim 1 wherein the apparatus includes a power supply to allow operation of the sensor.

12. Apparatus according to claim 1 wherein processing means are provided which include an integrated circuit with a micro controller unit to processes information from the said sensor and generate a corresponding response with reference to predetermined data.

13. Apparatus according to claim 11 wherein the processing means includes a wireless radio frequency transmitter which outputs responses from the micro controller unit.

14. Apparatus according to claim 11 wherein the processing means includes, or is connected to, an alarm unit which can be used to generate audible, visual and/or vibrational signals,

15. Apparatus according to claim 1 wherein the processing means track and analyse collected data and provides comparison with predetermined data and/or previously collected data.

16. Apparatus according to claim 11 wherein the processing means provides different forms of feedback to be selected to suit a person's and/or a third party's requirements, and these may include any, or any combination of; an instant/delayed reminder when the user has adopted a posture with inclinations/neck-flexions beyond the predetermined range in order to make the person rectify the incorrect position, and/or positive confirmation when the person has adopted the correct position in order to assist them in maintaining the correct position.

17. Apparatus according to claim 1 wherein a number of sensors are provided in order to allow head inclinations and neck-flexions on orthogonal axes to be determined and monitored.

18. (canceled)

19. Apparatus according to claim 1 wherein the apparatus provides medical support, such as to prevent spinal disease by ensuring that the person adopts the correct posture and/or assists in the confidence of the person by allowing the person to adopt a correct sitting posture.
